(19) United States
(12) Patent Application Publication
(10) Pub. No.: US 2005/0019279 A1
(43) Pub. Date: Jan. 27, 2005

(54) Cosmetic or dermatological preparation containing an octadecene dioic acid and a UV filtering substance

(75) Inventors: Anja Goppel, Hamburg (DE); Claudia Mundt, Bremen (DE); Jens Schulz, Schenefeld (DE); Rainer Wolber, Hamburg (DE); Thomas Blatt, Wedel (DE); Reza Keyhani, Hamburg (DE); Christoph Smuda, Bonningstedt (DE); Franz Stab, Echem (DE)

Correspondence Address: ALSTON & BIRD LLP;BANK OF AMERICA PLAZA, 101 SOUTH TRYON STREET, SUITE 4000, CHARLOTTE, NC, 28280-4000, US

(73) Assignee: Beiersdorf AG

(21) Appl. No.: 10/840,961

(22) Filed: May 7, 2004

Related U.S. Application Data
(63) Continuation of application No. PCT/EP02/12267, filed on Nov. 4, 2002.

(30) Foreign Application Priority Data
Nov. 9, 2001 (DE) 101 55 959.3
Nov. 9, 2001 (DE) 101 55 961.5

(51) Int. Cl. A61K 7/42

(52) U.S. Cl. 424/59

(57) ABSTRACT

The present invention is a cosmetic or dermatological preparation comprising at least one UV filtering substance and 8-hexadecene-1,16-dicarboxylic acid. The present invention also includes a method of using the preparation to treat or prevent undesired skin aging, skin pigmentation, or skin pigment disorders, and a method of using the preparation to lighten or prevent undesired pigmentation of the hair.

### CROSS-REFERENCE TO RELATED APPLICATIONS

This is a continuation application of PCT/EP02/12267, filed Nov. 4, 2002, which is incorporated herein by reference in its entirety, and also claims the benefit of German Priority Application Nos. 101 55 959.3 and 101 55 961.5, filed Nov. 9, 2001.

### FIELD OF THE INVENTION

The present invention relates to the use of active ingredients known per se for cosmetic and topical dermatological skin lightening or for preventing skin tanning, in particular skin tanning caused by UV radiation, and for lightening the natural color of hair.

### BACKGROUND OF THE INVENTION

In a preferred embodiment, the present invention relates to cosmetic and dermatological preparations for the prophylaxis and treatment of cosmetic or dermatological changes in skin, such as, for example, undesired pigmentation, for example local hyperpigmentation and incorrect pigmentation (for example liver spots, freckles), the inhibition of natural pigmentation, or for the purely cosmetic lightening of relatively large areas of skin which are quite appropriately pigmented for the individual skin type.

Pigmenting of the skin is caused by melanocytes, which are found in the lowest layer of the epidermis, the Stratum basal, alongside the basal cells as pigment-forming cells which, depending on the skin type, occur either individually or in clusters of varying size. Melanocytes contain, as characteristic cell organelles, melanosomes in which the melanin is formed. Among other things, excitation by UV radiation increases the formation of melanin. This melanin is transported via the living layers of the epidermis (keratinocytes) ultimately to the horny layer (corneocytes) and brings about a more or less marked brownish to brown-black skin color. Melanin is formed as the end stage of an oxidative process in which tyrosine, with co-action of the enzyme tyrosinase, is converted via several intermediates to the brown to brown-black eumelanins (DHICA and DHI melanin) or, with participation of sulfur-containing compounds, to the reddish pheomelanin. DHICA and DHI melanin are produced via the common intermediates dopaquinone and dopachrome. The latter, sometimes with participation of further enzymes, is converted into indole-5,6-quinonecarboxylic acid or into indole-5,6-quinone, from which the two eumelanins mentioned arise. The formation of pheomelanin proceeds inter alia by the intermediates dopaquinone and cysteinyldopa.

Similarly to the pigmenting of the skin, melanin-producing melanocytes are also responsible for the hair color (pigmentation of the hair). The amount and composition of melanin in the hair determines the natural hair color, which is genetically determined.

Problems with skin hyperpigmentation have a wide variety of causes and are accompanying phenomena of many biological processes, for example UV radiation (e.g. freckles, Ephelides), genetic disposition, incorrect pigmentation of the skin during wound healing or scarring or skin aging (e.g. Lentigines seniles).

Active ingredients and preparations which counteract skin pigmentation are known. In practice, use is made essentially of preparations based on hydroquinone although, on the one hand, these only show their effect after application for several weeks and, on the other hand, application of them for an excessively long time is not always without risk, for toxicological reasons. The inhibition of tyrosinase with substances such as kojic acid, ascorbic acid and azelaic acid and derivatives thereof is also common, although it has cosmetic and dermatological disadvantages.

For lightening the color of hair, strongly prooxidative processes are mostly used, which can severely damage the hair. In particular, the bleaching of hair using hydrogen peroxide is known in this regard.

### SUMMARY OF THE INVENTION

To remedy these shortcomings was the object of the present invention.

8-Hexadecene-1,16-dicarboxylic acid (dioic acid, CAS number 20701-68-2; provisional INCl name Octadecenedioic acid) is a metabolic product of yeast cells of the Candida strain. It is characterized by the following structure:

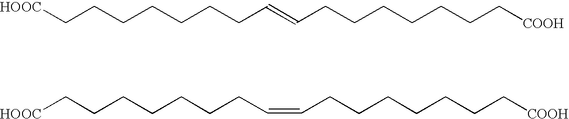

The starting substance used is a fatty acid of purely vegetable origin. This is converted to the hydroxy-fatty acid, which is then oxidized to the fatty acid aldehyde and ultimately to the dicarboxylic acid. The yeast cells originate from selected mutant strains. The commercial product has a purity of 95%. 8-hexadecene-1,16-dicarboxylic acid is here in the form of a mixture of the cis and trans isomers, the cis isomer being predominant in terms of quantity. Oleic acid may also be present in the product in a concentration of approximately 3%.

A large number of UV protective substances is used in cosmetic preparations for protecting the skin against sunlight and the damage to the skin caused thereby. These are inorganic pigments, UV-A, UV-B and/or broadband filtering substances.

Hydroxybenzophenones are characterized by the following structural formula: 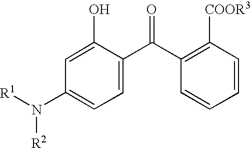
in which
R¹ and R², independently of one another, are hydrogen, C₁-C₂₀-alkyl, C₃-C₁₀-cycloalkyl or C₃-C₁₀-cycloalkenyl, where the substituents R¹ and R², together with the nitrogen atom to which they are bonded, can form a 5-membered or 6-membered ring and
R³ is a C₁-C₂₀-alkyl radical.

It was surprising and could not have been foreseen by the person skilled in the art that cosmetic or dermatological preparations containing UV filtering substances and comprising 8-hexadecene-1,16-dicarboxylic acid overcome the disadvantages of the prior art. In this connection, it is preferred when the preparations comprise UV filtering substances in concentrations of from 0.1% by weight to 30% by weight, preferably 0.5 to 20% by weight, particularly preferably 1.0 to 15.0% by weight, in each case based on the total weight of the preparations, and 8-hexadecene-1,16-dicarboxylic acid is present in a total concentration of 0.001-10% by weight, preferably 0.005-8% by weight, in particular 0.05-5% by weight, based on the total weight of the preparations. It is also preferred when a hydroxybenzophenone is used as UV filtering substance. It is also preferred when the hydroxybenzophenone chosen is hexyl 2-(4′-(diethylamino)-2′-hydroxybenzolyl)benzoate, which is characterized by the chemical structural formula

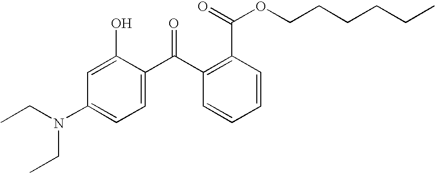

It is particularly advantageous when the content of one or more hydroxybenzophenones is from 0.1 to 20% by weight, preferably 0.1 to 15% by weight, particularly preferably 0.1 to 10% by weight, in each case based on the total weight of the preparations.

Furthermore, it is of great advantage when the preparations comprise at least one further UV filtering substance chosen from the group consisting of triazines, benzotriazoles, the UV filters liquid at room temperature and organic and/or inorganic pigments, preference being given here, as further UV-A filtering substance and/or broadband filter, to dibenzoylmethane derivatives [in particular 4-(tert-butyl)-4′-methoxydibenzoylmethane], phenylene-1,4-bis(2-benzimidazyl)-3,3′-5,5′-tetrasulfonic acid bis-sodium salt, 1,4-di(2-oxo-10-sulfo-3-bornylidenemethyl)benzene and salts thereof and 2,4-bis{[4-(2-ethylhexyloxy)-2-hydroxy]phenyl}-6-(4-methoxyphenyl)-1,3,5-triazine, where the further filtering substances may in each case be present individually or in any combinations with one another.

The invention also covers the use of such cosmetic or dermatological preparations against undesired pigmentation of the skin and/or for the treatment of pigment disorders. In addition, as a result of said active ingredient combination, the undesired pigmentation of the hair is reduced, thereby resulting in a lightening of the hair.

It is advantageous to also use 8-hexadecene-1,16-dicarboxylic acid in the form of one of its enantiomers.

The combination of 8-hexadecene-1,16-dicarboxylic acid and UV filters, referred to below as “active ingredient combination according to the invention” has proven to be exceptionally effective against undesired pigmentation, in particular local hyperpigmentation, and against skin tanning caused by UV radiation, both preventively and also in the sense of a treatment. It is, however, also extremely advantageous according to the invention to use the active ingredient combination according to the invention or cosmetic or topical dermatological preparations with an effective content of active ingredient combination according to the invention for the cosmetic or dermatological treatment of undesired skin pigmentation, thus, for example, inhomogeneous pigmentation of aging skin, Lentigines seniles or postinflammatory hyperpigmentation.

### DETAILED DESCRIPTION OF THE PREFERRED EMBODIMENTS

The prophylaxis and the cosmetic or dermatological treatment with the active ingredient combination according to the invention or with the cosmetic or topical dermatological preparations with an effective content of active ingredient combination according to the invention takes place in the customary manner, namely by applying the active ingredient combination according to the invention or the cosmetic or topical dermatological preparations with an effective content of active ingredient combination according to the invention to the affected areas of skin. Surprisingly, it has been found that the active ingredient combination according to the invention satisfies the object underlying the invention. Advantageously, the active ingredient combination according to the invention can be incorporated into customary cosmetic and dermatological preparations, which may be in various forms. Thus, for example, they may be a solution, an emulsion of the water-in-oil (W/O) type or of the oil-in-water (O/W) type, or multiple emulsions, for example of the water-in-oil-in-water (W/O/W) type or oil-in-water-in-oil (O/W/O) type, a hydrodispersion or lipodispersion, a gel, a solid stick, a transdermal therapeutic system, or an aerosol.

Emulsions according to the invention for the purposes of the present invention, for example in the form of a cream, a lotion, a cosmetic milk, are advantageous and comprise, for example, fats, oils, waxes and/or other fatty substances, and water and one or more emulsifiers, as are customarily used for such a type of formulation.

It is also possible and advantageous for the purposes of the present invention to incorporate the active ingredient combination according to the invention into aqueous systems or surfactant preparations for cleansing the skin and the hair.

It is of course known to the person skilled in the art that high-quality cosmetic compositions are in most cases inconceivable without customary auxiliaries and additives. These include, for example, consistency-imparting agents, fillers, perfume, dyes, emulsifiers, additional active ingredients, such as vitamins or proteins, photoprotective agents, stabilizers, insect repellants, alcohol, water, salts, antimicrobially, proteolytically or keratolytically effective substances, etc.

It is likewise advantageous to use the active ingredient 8-hexadecene-1,16-dicarboxylic acid in the form of molecular adducts on cyclodextrins. It is assumed that the cyclodextrin backbones here act as host molecule, and 8-hexadecene-1,16-dicarboxylic acid acts as guest molecule. For the preparation, cyclodextrins are dissolved in water and 8-hexadecene-1,16-dicarboxylic acid is added. The molecular adduct then precipitates out in the form of a solid and may be subjected to customary purification and work-up steps. It is known that cyclodextrin guest complexes are in an equilibrium in a corresponding solvent (e.g. water) between the concrete guest cyclodextrin complex and the dissociated form, it being possible to separate cyclodextrin and guest to a certain degree. Such equilibrium systems are likewise advantageous for the purposes of the present invention.

Corresponding requirements apply mutatis mutandis to the formulation of medicinal preparations.

For the purposes of the present invention, medicinal topical compositions generally comprise one or more medicaments in effective concentrations. For the sake of simplicity, for a clear distinction between cosmetic and medicinal use and corresponding products, reference is made to the legal provisions of the Federal Republic of Germany (e.g. Cosmetics Directive, Foods and Drugs Act).

In this connection, it is likewise advantageous to add the active ingredient combination according to the invention as additive to preparations which already comprise other active ingredients for other purposes.

Accordingly, cosmetic or topical dermatological compositions for the purposes of the present invention may, depending on their formulation, be used, for example, in the form of a skin protection cream, cleansing milk, sunscreen lotion, nourishing cream, day or night cream etc. It is in some instances possible and advantageous to use the compositions according to the invention as a base for pharmaceutical formulations.

It is also advantageous for the purposes of the present invention to provide cosmetic and dermatological preparations whose main purpose is not protection against sunlight, but which nevertheless contain a content of further UV protection substances. Thus, for example, UV-A and/or UV-B filtering substances are usually incorporated into day creams or makeup products. UV protection substances, like antioxidants and, if desired, preservatives, also represent effective protection of the preparations themselves against spoilage. Also favorable are cosmetic and dermatological preparations which are in the form of a sunscreen composition.

Accordingly, the preparations for the purposes of the present invention preferably comprise at least one further UV-A, UV-B and/or broadband filtering substance. The formulations can, but do not necessarily, optionally also comprise one or more organic and/or inorganic pigments as UV filtering substances which may be present in the water phase and/or the oil phase.

In addition, the preparations according to the invention can also advantageously be in the form of so-called oil-free cosmetic or dermatological emulsions which comprise a water phase and at least one UV filtering substance which is liquid at room temperature as a further phase.

Particularly advantageous UV filtering substances which are liquid at room temperature for the purposes of the present invention are homomenthyl salicylate (INCl: Homosalate), 2-ethylhexyl 2-cyano-3,3-diphenylacrylate (INCl: Octocrylene), 2-ethylhexyl 2-hydroxybenzoate (2-ethylhexyl salicylate, octyl salicylate, INCl: Octyl Salicylate) and esters of cinnamic acid, preferably 2-ethylhexyl 4-methoxycinnamate (INCl: Octyl Methoxycinnamate) and isopentyl 4-methoxycinnamate (INCl: Isoamyl p-Methoxycinnamate).

Preferred inorganic pigments are metal oxides and/or other metal compounds which are sparingly soluble or insoluble in water, in particular oxides of titanium (TiO₂), zinc (ZnO), iron (e.g. Fe₂O₃), zirconium (ZrO₂), silicon (SiO₂), manganese (e.g. MnO), aluminum (Al₂O₃), cerium (e.g. Ce₂O₃), mixed oxides of the corresponding metals, and mixtures of such oxides, and the sulfate of barium (BaSO₄).

For the purposes of the present invention, the pigments may advantageously also be used in the form of commercially available oily or aqueous predispersions. Dispersion auxiliaries and/or solubilization promoters may advantageously be added to these predispersions.

According to the invention, the pigments may advantageously be surface-treated (“coated”), the intention being, for example, to form or retain a hydrophilic, amphiphilic or hydrophobic character. This surface treatment can consist in providing the pigment with a thin hydrophilic and/or hydrophobic inorganic and/or organic layer by methods known per se. The different surface coatings may also comprise water for the purposes of the present invention.

Inorganic surface coatings for the purposes of the present invention may consist of aluminum oxide (Al₂O₃), aluminum hydroxide Al(OH)₃, and aluminum oxide hydrate (also: alumina, CAS No.: 1333-84-2), sodium hexametaphosphate (NaPO₃)₆, sodium metaphosphate (NaPO₃)ₙ, silicon dioxide (SiO₂) (also: silica, CAS No.: 7631-86-9), or iron oxide (Fe₂O₃). These inorganic surface coatings can occur on their own, in combination and/or in combination with organic coating materials.

Organic surface coatings for the purposes of the present invention can consist of vegetable or animal aluminum stearate, vegetable or animal stearic acid, lauric acid, dimethylpolysiloxane (also: dimethicone), methylpolysiloxane (methicone), simethicone (a mixture of dimethylpolysiloxane with an average chain length of from 200 to 350 dimethylsiloxane units and silica gel) or alginic acid. These organic surface coatings may exist on their own, in combination and/or in combination with inorganic coating materials.

Zinc oxide particles and predispersions of zinc oxide particles suitable according to the invention are available under the following trade names from the companies listed:

| Trade name | Coating | Manufacturer |
|---|---|---|
| Z-Cote HP1 | 2% dimethicone | BASF |
| Z-Cote | / | BASF |
| ZnO NDM | 5% dimethicone | H&R |

Suitable titanium dioxide particles and predispersions of titanium dioxide particles are available under the following trade names from the companies listed:

| Trade name | Coating | Manufacturer |
|---|---|---|
| MT-100TV | Aluminum hydroxide/stearic | Tayca Corporation |
| | acid | |
| MT-100Z | Aluminum hydroxide/stearic | Tayca Corporation |
| | acid | |
| Eusolex T-2000 | Alumina/simethicone | Merck KGaA |
| Titanium dioxide | Octyltrimethylsilane | Degussa |
| T805 | | |
| (Uvinul TiO₂) | | |

Advantageous UV-A filtering substances for the purposes of the present invention are dibenzoylmethane derivatives, in particular 4-(tert-butyl)-4′-methoxydibenzolymethane (CAS No. 70356-09-1), which is sold by Givaudan under the name Parsol® 1789 and by Merck under the trade name Eusolex® 9020.

Advantageous further UV filtering substances for the purposes of the present invention are sulfonated, water-soluble UV filters, such as, for example: phenylene-1,4-bis(2-benzimidazyl)-3,3′-5,5′-tetrasulfonic acid and its salts, particularly the corresponding sodium, potassium or triethanolammonium salts, in particular the phenylene-1,4-bis(2-benzimidazyl)-3,3′-5,5′-tetrasulfonic acid bis-sodium salt with the INCl name Bisimidazylate (CAS No.: 180898-37-7), which is available, for example, under the trade name Neo Heliopan AP from Haarmann & Reimer;
salts of 2-phenylbenzimidazole-5-sulfonic acid, such as its sodium, potassium, or its triethanolammonium salt, and the sulfonic acid itself with the INCl name Phenylbenzimidazole Sulfonic Acid (CAS No. 27503-81-7), which is available, for example, under the trade name Eusolex 232 from Merck or under Neo Heliopan Hydro from Haarmann & Reimer;
1,4-di(2-oxo-10-sulfo-3-bornylidenemethyl)benzene (also: 3,3′-(1,4-phenylenedimethylene)bis(7,7-dimethyl-2-oxobicyclo[2.2.1]hept-1-ylmethanesulfonic acid) and salts thereof (particularly the corresponding 10-sulfato compounds, in particular the corresponding sodium, potassium or triethanolammonium salt), which is also referred to as benzene-1,4-di(2-oxo-3-bornylidenemethyl-10-sulfonic acid). Benzene-1,4-di(2-oxo-3-bornylidenemethyl-10-sulfonic acid) has the INCl name Terephthalidene Dicamphor Sulfonic Acid (CAS No.: 90457-82-2) and is available, for example, under the trade name Mexoryl SX from Chimex;
sulfonic acid derivatives of 3-benzylidenecamphor, such as, for example, 4-(2-oxo-3-bornylidenemethyl)benzenesulfonic acid, 2-methyl-5-(2-oxo-3-bornylidenemethyl)sulfonic acid and salts thereof.

Advantageous UV filtering substances for the purposes of the present invention are also so-called broadband filters, i.e. filtering substances which absorb both UV-A and UV-B radiation.

Advantageous broadband filters or UV-B filtering substances are, for example, triazine derivatives, such as, for example,
2,4-bis{[4-(2-ethylhexyloxy)-2-hydroxy]phenyl}-6-(4-methoxyphenyl)-1,3,5-triazine (INCl: Bis-Ethylhexyloxyphenol Methoxyphenyl Triazine (BEMT)), which is available under the trade name Tinosorb® S from CIBA-Chemikalien GmbH;
diethylhexylbutylamidotriazone (INCl: Diethylhexylbutamidotriazone), which is available under the trade name UVASORB HEB from Sigma 3V;
tris(2-ethylhexyl) 4,4′,4″-(1,3,5-triazine-2,4,6-triyltriimino)trisbenzoate, also: 2,4,6-tris[anilino(p-carbo-2′-ethyl-1′-hexyloxy)]-1,3,5-triazine (INCl: Ethylhexyl Triazone), which is sold by BASF Aktiengesellschaft under the trade name UVINUL® T 150.

An advantageous broadband filter for the purposes of the present invention is also 2,2′-methylenebis(6-(2H-benzotriazol-2-yl)-4-(1,1,3,3-tetramethylbutyl)phenol), which is available under the trade name Tinosorb® M from CIBA-Chemikalien GmbH.

Another advantageous broadband filter for the purposes of the present invention is 2-(2H-benzotriazol-2-yl)-4-methyl-6-[2-methyl-3-[1,3,3,3-tetramethyl-1-[(trimethylsilyl)oxy]disiloxanyl]propyl]phenol (CAS No.: 155633-54-8) with the INCl Name Drometrizole Trisiloxane, which is available under the trade name Mexoryl® XL from Chimex.

The further UV filtering substances may be oil-soluble or water-soluble.

Advantageous oil-soluble UV-B and/or broadband filtering substances for the purposes of the present invention are, for example:
3-benzylidenecamphor derivatives, preferably 3-(4-methylbenzylidene)camphor, 3-benzylidenecamphor;
4-aminobenzoic acid derivatives, preferably 2-ethylhexyl 4-(dimethylamino)benzoate, amyl 4-(dimethylamino)benzoate;
derivatives of benzophenone, preferably 2-hydroxy-4-methoxybenzophenone, 2-hydroxy4-methoxy-4′-methylbenzophenone, 2,2′-dihydroxy-4-methoxy-benzophenone;
and UV filters bonded to polymers;
3-(4-(2,2-bis-ethoxycarbonylvinyl)phenoxy)propenyl)methoxysiloxane/dimethyl-siloxane-copolymer which is available, for example, under the trade name Parsol® SLX from Hoffmann La Roche.

Advantageous water-soluble filtering substances are, for example:
Sulfonic acid derivatives of 3-benzylidenecamphor, such as, for example, 4-(2-oxo-3-bornylidenemethyl)benzenesulfonic acid, 2-methyl-5-(2-oxo-3-bornylidenemethyl)sulfonic acid and salts thereof.

A further photoprotective filtering substance to be used advantageously according to the invention is ethylhexyl 2-cyano-3,3-diphenylacrylate (octocrylene), which is available from BASF under the name Uvinul® N 539.

Besides comprising the filtering substance(s) according to the invention, particularly advantageous preparations for the purposes of the present invention which are characterized by high or very high UV-A and/or UV-B protection preferably also comprise further UV-A and/or broadband filters, in particular dibenzoylmethane derivatives [for example 4-(tert-butyl)-4′-methoxydibenzoylmethane], phenylene-1,4-bis(2-benzimidazyl)-3,3′,5,5′-tetrasulfonic acid and/or its salts, 1,4-di(2-oxo-10-sulfo-3-bornylidenemethyl)benzene and/or salts thereof and/or 2,4-bis{[4-(2-ethylhexyloxy)-2-hydroxy]phenyl}-6-(4-methoxyphenyl)-1,3,5-triazine, in each case individually or in any combinations with one another.

The list of specified UV filters which can be used for the purposes of the present invention is not intended to be limiting.

Advantageously, the preparations according to the invention comprise the substances which absorb UV radiation in the UV-A and/or UV-B region in a total amount of, for example, 0.1% by weight to 30% by weight, preferably 0.5 to 20% by weight, in particular 1.0 to 15.0% by weight, in each case based on the total weight of the preparations, in order to provide cosmetic preparations which protect the hair and/or the skin from the entire range of ultraviolet radiation.

The cosmetic and dermatological preparations according to the invention can comprise cosmetic active ingredients, auxiliaries and/or additives, as are customarily used in such preparations, e.g. antioxidants, preservatives, bactericides, perfumes, antifoams, dyes, pigments which have a coloring effect, thickeners, surface-active substances, emulsifiers, softening, moisturizing and/or humectant substances, fats, oils, waxes, or other customary constituents of a cosmetic or dermatological formulation, such as alcohols, polyols, polymers, foam stabilizers, electrolytes, organic solvents or silicone derivatives.

It is likewise advantageous to add customary antioxidants to the preparations for the purposes of the present invention. According to the invention, all antioxidants which are customary or suitable for cosmetic and dermatological applications may be used as favorable antioxidants.

The antioxidants are advantageously chosen from the group consisting of amino acids (e.g. glycine, histidine, tyrosine, tryptophan) and derivatives thereof, imidazoles (e.g. urocanic acid) and derivatives thereof, peptides, such as D,L-carnosine, D-carnosine, L-carnosine and derivatives thereof (e.g. anserine), carotenoids, carotenes (e.g. α-carotene, β-carotene, lycopene) and derivatives thereof, retinoids, such as, for example, retinol, retinal and/or retinoic acid and the respective esters, α-lipoic acid and derivatives thereof (e.g. dihydrolipoic acid), aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (e.g. buthionine sulfoximines, homocysteine sulfoximine, buthionine sulfones, penta-, hexa-, heptathionine sulfoximine) in very low tolerated doses (e.g. pmol to μmol/kg), and also (metal) chelating agents (e.g. α-hydroxy fatty acids, palmitic acid, phytic acid, lactoferrin), α-hydroxy acids (e.g. citric acid, lactic acid, maleic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, 2-aminopropionic acid diacetic acid, flavonoids, polyphenols, catechins, ubiquinone and ubiquinol and derivatives thereof, vitamin C and derivatives (e.g. ascorbyl palmitate, Mg ascorbyl phosphate, ascorbyl acetate), tocopherols and derivatives (e.g. vitamin E acetate), and coniferyl benzoate of benzoin resin, rutinic acid and derivatives thereof, ferulic acid and derivatives thereof, butylhydroxytoluene, butylhydroxyanisole, nordihydroguaiacic acid, nordihyrdoguaiaretic acid, trihydroxybutyrophenone, uric acid and derivatives thereof, mannose and derivatives thereof, zinc and derivatives thereof, (e.g. ZnO, ZnSO₄), selenium and derivatives thereof (e.g. selenomethionine), stilbene and derivatives thereof (e.g. silbene oxide, trans-stilbene oxide) and the derivatives (salts, esters, ethers, sugars, nucleotides, nucleosides, peptides and lipids) of said active ingredients which are suitable according to the invention.

The amount of antioxidants (one or more compounds) in the preparations is preferably 0.001 to 30% by weight, particularly preferably 0.05 to 20% by weight, in particular 0.1 to 10% by weight, based on the total weight of the preparation.

If vitamin E and/or derivatives thereof are the antioxidant or antioxidants, it is advantageous to choose their respective concentrations from the range 0.001 to 10% by weight, based on the total weight of the formulation.

If the cosmetic or dermatological preparation for the purposes of the present invention is a solution or emulsion or dispersion, the solvents used may be:
water or aqueous solutions
oils, such as triglycerides of capric acid or of caprylic acid, but preferably castor oil;
fats, waxes and other natural and synthetic fatty substances, preferably esters of fatty acids with alcohols of low carbon number, e.g. with isopropanol, propylene glycol or glycerol, or esters of fatty alcohols with alkanoic acids of low carbon number or with fatty acids;
alcohols, diols or polyols of low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monethyl ether and analogous products.

In particular, mixtures of the abovementioned solvents are used. In the case of alcoholic solvents, water may be a further constituent.

The oil phase of the emulsions, oleogels or hydrodispersions or lipodispersions for the purposes of the present invention is advantageously chosen from the group of esters of saturated and/or unsaturated, branched and/or unbranched alkanecarboxylic acids with a chain length of from 3 to 30 carbon atoms and saturated and/or unsaturated, branched and/or unbranched alcohols with a chain length of from 3 to 30 carbon atoms, from the group of esters of aromatic carboxylic acids and saturated and/or unsaturated, branched and/or unbranched alcohols with a chain length of from 3 to 30 carbon atoms. Such ester oils can then advantageously be chosen from the group consisting of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate, and synthetic, semisynthetic and natural mixtures of said esters, e.g. jojoba oil.

In addition, the oil phase can advantageously be chosen from the group of branched and unbranched hydrocarbons and hydrocarbon waxes, silicone oils, dialkyl ethers, the group of saturated or unsaturated, branched or unbranched alcohols, and fatty acid triglycerides, namely the triglycerol esters of saturated and/or unsaturated, branched and/or unbranched alkanecarboxylic acids with a chain length of from 8 to 24, in particular 12-18, carbon atoms. The fatty acid triglycerides can, for example, be chosen advantageously from the group of synthetic, semisynthetic and natural oils, e.g. olive oil, sunflower oil, soybean oil, peanut oil, rapeseed oil, almond oil, palm oil, coconut oil, palm kernel oil and the like.

Any mixtures of such oil and wax components are also to be used advantageously for the purposes of the present invention. It may in some instances also be advantageous to use waxes, for example cetyl palmitate, as the sole lipid component of the oil phase.

The oil phase is advantageously chosen from the group consisting of 2-ethylhexyl isostearate, octyldodecanol, isotridecyl isononanoate, isoeicosane, 2-ethylhexyl cocoate, C₁₂₋₁₅-alkyl benzoate, caprylic/capric acid triglyceride, dicaprylyl ether.

Mixtures of C₁₂₋₁₅-alkyl benzoate and 2-ethylhexyl isostearate, mixtures of C₁₂₋₁₅-alkyll benzoate and isotridecyl isononanoate, and mixtures of C₁₂₋₁₅-alkyl benzoate, 2-ethylhexyl isostearate and isotridecyl isononanoate are particularly advantageous.

Of the hydrocarbons, paraffin oils, squalane and squalene are to be used advantageously for the purposes of the present invention.

Advantageous oil components are also, for example, butyloctyl salicylate (for example that available under the trade name Hallbrite BHB from CP Hall), hexadecyl benzoate and butyloctyl benzoate and mixtures thereof (Hallstar AB) and/or diethylhexyl naphthalate (Hallbrite TQ).

The oil phase can also advantageously have a content of cyclic or linear silicone oils, or consist entirely of such oils, although it is preferred to use an additional content of other oil phase components apart from the silicone oil or the silicone oils.

Advantageously, cyclomethicone (octamethylcyclotetrasiloxane) is used as silicone oil to be used according to the invention. However, other silicone oils can also be used advantageously for the purposes of the present invention, for example hexamethylcyclotrisiloxane, polydimethylsiloxane, poly(methylphenylsiloxane).

Mixtures of cyclomethicone and isotridecyl isononanoate, and of cyclomethicone and 2-ethylhexyl isostearate are also particularly advantageous.

Gels used according to the invention usually comprise alcohols of low carbon number, e.g. ethanol, isopropanol, 1,2-propanediol, glycerol and water or an abovementioned oil in the presence of a thickener, which in the case of oily-alcoholic gels is preferably silicon dioxide or an aluminum silicate, and in the case of aqueous-alcoholic or alcoholic gels is preferably a polyacrylate.

Solid sticks comprise, for example, natural or synthetic waxes, fatty alcohols or fatty acid esters. Preference is given to using lip care sticks, and stick formulations for deodorizing the body.

Customary basic substances which are suitable for use as cosmetic sticks for the purposes of the present invention are liquid oils (e.g. paraffin oils, castor oil, isopropyl myristate), semisolid constituents (e.g. petroleum jelly, lanolin), solid constituents (e.g. beeswax, ceresin and microcrystalline waxes and ozocerite), and high-melting waxes (e.g. carnauba wax, candelilla wax).

Suitable propellants for cosmetic and/or dermatological preparations for the purposes of the present invention which can be sprayed from aerosol containers are the customary known readily volatile, liquefied propellants, for example hydrocarbons (propane, butane, isobutane), which can be used on their own or in a mixture with one another. Compressed air can also be used advantageously.

The person skilled in the art is of course aware that there are propellant gases which are nontoxic per se which would in principle be suitable for realizing the present invention in the form of aerosol preparations, but which nevertheless have to be avoided due to a harmful effect on the environment or other accompanying phenomena, in particular fluorocarbons and chlorofluorocarbons (CFCs).

Cosmetic preparations for the purposes of the present invention may also be in the form of gels which, besides an effective content of active ingredient according to the invention and solvents customarily used therefor, preferably water, also comprise organic thickeners, e.g. gum arabic, xanthan gum, sodium alginate, cellulose derivatives, preferably methylcellulose, hydroxymethylcellulose, hydroxyethylcellulose, hydroxypropylcellulose, hydroxpropylmethylcellulose or inorganic thickeners, e.g. aluminum silicates, such as, for example, bentonites, or a mixture of polyethylene glycol and polyethylene glycol stearate or distearate. The thickener is present in the gel, for example, in an amount between 0.1 and 30% by weight, preferably between 0.5 and 15% by weight.

The examples below are intended to illustrate the present invention.

Unless stated otherwise, all amounts, percentages or parts refer to the total weight of the preparations or the particular mixtures. In the examples, “aminobenzophenone” should be understood as meaning hexyl 2-(4′-(diethylamino)-2′-hydroxybenzoyl)benzoate.

### FORMULATION EXAMPLES

PIT Emulsions

| | Examples | | | | |
|---|---|---|---|---|---|
| | 1 | 2 | 3 | 4 | 5 |
| Glycerol monostearate self- | 0.50 | | 3.00 | 2.00 | 4.00 |
| emulsifying | | | | | |
| Polyoxyethylene(12) cetylstearyl | | 5.00 | | 1.00 | 1.50 |
| ether | | | | | |
| Polyoxyethylene(20) cetylstearyl | | | | 2.00 | |
| ether | | | | | |
| Polyoxyethylene(30) cetylstearyl | 5.00 | | 1.00 | | |
| ether | | | | | |
| Stearyl alcohol | | | 3.00 | | 0.50 |
| Cetyl alcohol | 2.50 | 1.00 | | 1.50 | |
| 2-ethylhexyl methoxycinnamate | | | | 5.00 | 8.00 |
| 2,4-bis(4-(2-ethylhexyloxy)-2- | | 1.50 | | 2.00 | 2.50 |
| hydroxyl)phenyl)-6-(4- | | | | | |
| methoxyphenyl)(1,3,5)-triazine | | | | | |
| Butyldimethoxydibenzoylmethane | | | 2.00 | | |
| Diethylhexylbutamidotriazone | 1.00 | 2.00 | | 2.00 | |
| Ethylhexyltriazone | 4.00 | | 3.00 | 4.00 | |
| 4-Methylbenzylidenecamphor | | 4.00 | | | 2.00 |
| Octocrylene | | 4.00 | | | 2.50 |
| Phenylene-1,4-bis(monosodium, | | | 0.50 | | 1.50 |
| 2-benzimidazyl-5,7)-disulfonic acid | | | | | |
| Phenylbenzimidazole sulfonic acid | 0.50 | | | 3.00 | |
| C₁₂₋₁₅ alkyl benzoate | | 2.50 | | | 5.00 |
| Titanium dioxide | 0.50 | 1.00 | | 3.00 | 2.00 |
| Zinc oxide | 2.00 | | 3.00 | 0.50 | 1.00 |
| Dicaprylyl ether | | | 3.50 | | |
| Butylene glycol dicaprylate/dicaprate | 5.00 | | | 6.00 | |
| Dicaprylyl carbonate | | | 6.00 | | 2.00 |
| Dimethicone polydimethylsiloxane | | 0.50 | 1.00 | | |
| Phenylmethylpolysiloxane | 2.00 | | | 0.50 | 0.50 |
| Shea butter | | 2.00 | | | 0.50 |
| PVP hexadecene copolymer | 0.50 | | | 0.50 | 1.00 |
| Glycerol | 3.00 | 7.50 | 5.00 | 7.50 | 2.50 |
| Tocopherol acetate | 0.50 | | 0.25 | | 1.00 |
| Dioic acid | 0.2 | 1.1 | 0.3 | 0.8 | 0.5 |
| Alpha-glucosylrutin | 0.10 | | 0.20 | | |
| Preservative | q.s. | q.s. | q.s. | q.s. | q.s. |
| Ethanol | 3.00 | 2.00 | 1.50 | | 1.00 |
| Perfume | q.s. | q.s. | q.s. | q.s. | q.s. |
| Water | ad | ad | ad | ad | ad |
| | 100 | 100 | 100 | 100 | 100 |

O/W Emulsions

| | Examples | | | | |
|---|---|---|---|---|---|
| | 1 | 2 | 3 | 4 | 5 |
| Glyceryl stearate citrate | | | 2.00 | | 2.00 |
| Glyceryl stearate self-emulsifying | 4.00 | 3.00 | | | |
| PEG-40 stearate | 1.00 | | | | |
| Polyglyceryl-3 methylglucose distearate | | | | 3.00 | |
| Sorbitan stearate | | | | | 2.00 |
| Stearic acid | | 1.00 | | | |
| Stearyl alcohol | | | 5.00 | | |
| Cetyl alcohol | 3.00 | 2.00 | | 3.00 | |
| Cetyl stearyl alcohol | | | | | 2.00 |
| Caprylic/capric triglyceride | 5.00 | 3.00 | 4.00 | 3.00 | 3.00 |
| Octyldodecanol | | | 2.00 | | 2.00 |
| Dicaprylyl ether | | 4.00 | | 2.00 | 1.00 |
| Paraffinum liquidum | 5.00 | 2.00 | | 3.00 | |
| Titanium dioxide | | | 1.00 | | |
| Octocrylene | | | 3.50 | | |
| Butyldimethoxydibenzoylmethane | | | 0.50 | | |
| Dioic acid | 0.10 | 0.20 | 0.70 | 0.15 | 1.00 |
| Tocopherol | 0.10 | | | | 0.20 |
| Biotin | | | 0.05 | | |
| Ethylenediaminetetraacetic acid | 0.1 | | 0.10 | 0.1 | |
| trisodium | | | | | |
| Preservative | q.s. | q.s. | q.s. | q.s. | q.s. |
| Polyacrylic acid | 3.00 | 0.1 | | 0.1 | 0.1 |
| Sodium hydroxide solution 45% | q.s. | q.s. | q.s. | q.s. | q.s. |
| Glycerol | 5.00 | 3.00 | 4.00 | 3.00 | 3.00 |
| Butylene glycol | | 3.00 | | | |
| Perfume | q.s. | q.s. | q.s. | q.s. | q.s. |
| Water | ad 100 | ad 100 | ad 100 | ad 100 | ad 100 |
| | Examples | | | | |
| | 6 | 7 | 8 | 9 | 10 |
| Glyceryl stearate citrate | | 2.00 | 2.00 | | |
| Glyceryl stearate self-emulsifying | 5.00 | | | | |
| Stearic acid | | | | 2.50 | 3.50 |
| Stearyl alcohol | 2.00 | | | | |
| Cetyl alcohol | | | | 3.00 | 4.50 |
| Cetyl stearyl alcohol | | 3.00 | 1.00 | | 0.50 |
| C₁₂₋₁₅-alkyl benzoate | | 2.00 | 3.00 | | |
| Caprylic/capric triglyceride | 2.00 | | | | |
| Octyldodecanol | 2.00 | 2.00 | | 4.00 | 6.00 |
| Dicaprylyl ether | | | | | |
| Paraffinum liquidum | | 4.00 | 2.00 | | |
| Cyclic dimethylpolysiloxane | | | | 0.50 | 2.00 |
| Dimethicone polydimethylsiloxane | 2.00 | | | | |
| Titanium dioxide | 2.00 | | | | |
| 4-methylbenzylidenecamphor | 1.00 | | | | |
| Ethylhexyltriazone | | | | | 2.00 |
| Butyldimethoxydibenzoylmethane | 0.50 | | | | 0.50 |
| Dioic acid | 0.30 | 0.10 | 1.00 | 0.50 | 0.10 |
| Tocopherol | | | | | 0.10 |
| Ethylenediaminetetraacetic acid | | | 0.20 | | 0.20 |
| trisodium | | | | | |
| Preservative | q.s. | q.s. | q.s. | q.s. | q.s. |
| Xanthan gum | | | 0.20 | | |
| Polyacrylic acid | 0.15 | 0.1 | | 0.05 | 0.05 |
| Sodium hydroxide solution 45% | q.s. | q.s. | q.s. | q.s. | q.s. |
| Glycerol | 3.00 | | 3.00 | 5.00 | 3.00 |
| Butylene glycol | | 3.00 | | | |
| Ethanol | | 3.00 | | 3.00 | |
| Perfume | q.s. | qs. | qs. | qs. | q.s. |
| Water | ad 100 | ad 100 | ad 100 | ad 100 | ad 100 |

W/O Emulsions

| | Examples | | | | |
|---|---|---|---|---|---|
| | 1 | 2 | 3 | 4 | 5 |
| Cetyldimethicone copolyol | | 2.50 | | 4.00 | |
| Polyglyceryl-2 dipolyhydroxystearate | 5.00 | | | | 4.50 |
| PEG-30 dipolyhydroxystearate | | | 5.00 | | |
| 2-Ethylhexyl methoxycinnamate | | 8.00 | | 5.00 | 4.00 |
| 2,4-Bis(4-(2-ethylhexyloxy)-2- | 2.00 | 2.50 | | 2.00 | 2.50 |
| hydroxy)phenyl)-6-(4- | | | | | |
| methoxyphenyl)-(1,3,5)-triazine | | | | | |
| Butyldimethoxydibenzoylmethane | | | 2.00 | 1.00 | |
| Diethylhexylbutamidotriazone | 3.00 | 1.00 | | | 3.00 |
| Ethylhexyltriazone | | | 3.00 | 4.00 | |
| 4-Methylbenzylidenecamphor | | 2.00 | | 4.00 | 2.00 |
| Octocrylene | 7.00 | 2.50 | 4.00 | | 2.50 |
| Diethylhexylbutamidotriazone | 1.00 | | | 2.00 | |
| Phenylene-1,4-bis(monosodium, | 1.00 | 2.00 | 0.50 | | |
| 2-benzimidazyl-5,7-disulfonic acid) | | | | | |
| Phenylbenzimidazolesulfonic acid | 0.50 | | | 3.00 | 2.00 |
| Titanium dioxide | | 2.00 | 1.50 | | 3.00 |
| Zinc oxide | 3.00 | 1.00 | 2.00 | 0.50 | |
| Paraffinum liquidum | | | 10.00 | | 8.00 |
| C₁₂₋₁₅ alkyl benzoate | | | | 9.00 | |
| Dicaprylyl ether | 10.00 | | | | 7.00 |
| Butylene glycol dicaprylate/dicaprate | | | 2.00 | 8.00 | 4.00 |
| Dicaprylyl carbonate | 5.00 | | 6.00 | | |
| Dimethicone polydimethylsiloxane | | 4.00 | 1.00 | 5.00 | |
| Phenylmethylpolysiloxane | 2.00 | 25.00 | | | 2.00 |
| Shea Butter | | | 3.00 | | |
| PVP hexadecene copolymer | 0.50 | | | 0.50 | 1.00 |
| Octoxyglycerol | | 0.30 | 1.00 | | 0.50 |
| Glycerol | 3.00 | 7.50 | | 7.50 | 2.50 |
| Glycine soya | | 1.00 | 1.50 | | |
| Magnesium sulfate | 1.00 | 0.50 | | 0.50 | |
| Magnesium chloride | | | 1.00 | | 0.70 |
| Tocopherol acetate | 0.50 | | 0.25 | | 1.00 |
| Dioic acid | 0.2 | 0.3 | 0.6 | 1 | 1.5 |
| Preservative | q.s. | q.s. | q.s. | q.s. | q.s. |
| Ethanol | 3.00 | | 1.50 | | 1.00 |
| Perfume | q.s. | qs. | qs. | qs. | q.s. |
| Water | ad 100 | ad 100 | ad 100 | ad 100 | ad 100 |

Examples

| | 6 | 7 |
|---|---|---|
| Polyglyceryl-2 dipolyhydroxystearate | 4.00 | 5.00 |
| Lanolin alcohol | 0.50 | 1.50 |
| Isohexadecane | 1.00 | 2.00 |
| Myristyl myristate | 0.50 | 1.50 |
| Petroleum jelly | 1.00 | 2.00 |
| Butyldimethoxydibenzoylmethane | 0.50 | 1.50 |
| 4-Methylbenzylidenecamphor | 1.00 | 3.00 |
| Butylene glycol dicaprylate/dicaprate | 4.00 | 5.00 |
| Shea butter | | 0.50 |
| Butylene glycol | | 6.00 |
| Octoxyglycerol | | 3.00 |
| Glycerol | 5.00 | |
| Tocopherol acetate | 0.50 | 1.00 |
| Dioic acid | 0.50 | 0.20 |
| Trisodium EDTA | 0.20 | 0.20 |
| Preservative | q.s. | q.s. |
| Ethanol | | 3.00 |
| Perfume | q.s. | q.s. |
| Water | ad 100 | ad 100 |

Hydrodispersions

| | Examples | | | | |
|---|---|---|---|---|---|
| | 1 | 2 | 3 | 4 | 5 |
| Polyoxyethylene(20) cetylstearyl | 1.00 | | | 0.5 | |
| ether | | | | | |
| Cetyl alcohol | | | 1.00 | | |
| Sodium polyacrylate | | 0.20 | | 0.30 | |
| Acrylates/C10-30-alkyl acrylate | 0.50 | | 0.40 | 0.10 | 0.10 |
| crosspolymer | | | | | |
| Xanthan gum | | 0.30 | 0.15 | | 0.50 |
| 2-Ethylhexyl methoxycinnamate | | | | 5.00 | 8.00 |
| 2,4-Bis(4-(2-ethylhexyloxy)-2- | | 1.50 | | 2.00 | 2.50 |
| hydroxy)phenyl)-6-(4- | | | | | |
| methoxyphenyl)(1,3,5)triazine | | | | | |
| Butyldimethoxydibenzoylmethane | 1.00 | | 2.00 | | |
| Diethylhexylbutamidotriazone | | 2.00 | | 2.00 | 1.00 |
| Ethylhexyltriazone | 4.00 | | 3.00 | 4.00 | |
| 4-Methylbenzylidenecamphor | 4.00 | 4.00 | | | 2.00 |
| Octocrylene | | 4.00 | 4.00 | | 2.50 |
| Phenylene-1,4-bis(monosodium, | 1.00 | | 0.50 | | 2.00 |
| 2-benzimidazyl-5,7-disulfonic acid) | | | | | |
| Phenylbenzimidazolesulfonic acid | 0.50 | | | 3.00 | |
| Titanium dioxide | 0.50 | | 2.00 | 3.00 | 1.00 |
| Zinc oxide | 0.50 | 1.00 | 3.00 | | 2.00 |
| C₁₂₋₁₅ alkyl benzoate | 2.00 | 2.50 | | | |
| Dicaprylyl ether | | 4.00 | | | |
| Butylene glycol dicaprylate/dicaprate | 4.00 | | 2.00 | 6.00 | |
| Dicaprylyl carbonate | | 2.00 | 6.00 | | |
| Dimethicone polydimethylsiloxane | | 0.50 | 1.00 | | |
| Phenylmethylpolysiloxane | 2.00 | | | 0.50 | 2.00 |
| Shea butter | | 2.00 | | | |
| PVP hexadecene copolymer | 0.50 | | | 0.50 | 1.00 |
| Octoxyglycerol | | | 1.00 | | 0.50 |
| Glycerol | 3.00 | 7.50 | | 7.50 | 2.50 |
| Glycine soya | | | 1.50 | | |
| Tocopherol acetate | 0.50 | | 0.25 | | 1.00 |
| Dioic acid | 0.4 | 0.2 | 0.6 | 0.5 | 1 |
| Preservative | q.s. | q.s. | q.s. | q.s. | q.s. |
| Ethanol | 3.00 | 2.00 | 1.50 | | 1.00 |
| Perfume | q.s. | qs. | qs. | qs. | q.s. |
| Water | ad | ad | ad | ad | ad |
| | 100 | 100 | 100 | 100 | 100 |

Gel Cream:

| | |
|---|---|
| Acrylate/C10-30 alkyl acrylate crosspolymer | 0.40 |
| Polyacrylic acid | 0.20 |
| Xanthan gum | 0.10 |
| Cetearyl alcohol | 3.00 |
| C₁₂₋₁₅ alkyl benzoate | 4.00 |
| Caprylic/capric triglyceride | 3.00 |
| Ethylhexyl methoxycinnamate | 5.00 |
| Bis-ethylhexyloxyphenol | 1.00 |
| methoxyphenyltriazine | |
| Ethylhexyltriazone | 2.00 |
| Cyclic dimethylpolysiloxane | 5.00 |
| Dimethicone polydimethylsiloxane | 1.00 |
| Dioic acid | 1.00 |
| Glycerol | 3.00 |
| Sodium hyroxide | q.s. |
| Preservative | q.s. |
| Perfume | q.s. |
| Water | ad 100.0 |

pH adjusted to 6.0

W/O Creams

| | |
|---|---|
| Polyglyceryl-3 diisostearate | 3.50 |
| Glycerol | 3.00 |
| Polyglyceryl-2-dipolyhydroxystearate | 3.50 |
| Dioic acid | 1 |
| Preservative | q.s. |
| Ethylhexyl methoxycinnamate | 5.00 |
| Bis-ethylhexyloxyphenol | 0.80 |
| methoxyphenyltriazine | |
| Octocrylene | 3.00 |
| Perfume | q.s. |
| Water | ad 100.0 |
| Magnesium sulfate | 0.6 |
| Isopropyl stearate | 2.0 |
| Caprylyl ether | 8.0 |
| Cetearyl isononanoate | 6.0 |

W/O/W Cream

| | |
|---|---|
| Glyceryl stearate | 3.00 |
| PEG-100 stearate | 0.75 |
| Behenyl alcohol | 2.00 |
| Caprylic/capric triglyceride | 8.00 |
| Octyldodecanol | 5.00 |
| C₁₂₋₁₅ alkyl benzoate | 3.00 |
| Dioic acid | 1 |
| Ethylhexyl methoxycinnamate | 5.00 |
| Bis-ethylhexyloxyphenol methoxyphenyltriazine | 1.80 |
| Ethylhexyltriazone | 1.50 |
| Magnesium sulfate (MgSO₄) | 0.80 |
| Ethylenediaminetetraacetic acid | 0.10 |
| Preservative | q.s. |
| Perfume | q.s. |
| Water | ad 100.0 |

pH adjusted to 6.0

Conditioner Shampoo with Pearlescence

| | Examples | | |
|---|---|---|---|
| | 1 | 2 | 3 |
| Polyquaternium-10 | 0.5 | 0.5 | 0.5 |
| Sodium laureth sulfate | 9.0 | 9.0 | 9.0 |
| Cocoamidopropylbetaine | 2.5 | 2.5 | 2.5 |
| Benzophenone-3 | 1.5 | 0.5 | 1.00 |
| Pearlizing agent | 2.0 | 2.0 | 2.0 |
| Dioic acid | 0.1 | 0.15 | 0.05 |
| Disodium EDTA | 0.1 | 0.2 | 0.15 |
| Preservative, perfume, thickener, | q.s. | q.s. | q.s. |
| pH adjustment and solubility | | | |
| promoter | | | |
| Water (demineralized) | ad | ad | ad |
| | 100.0 | 100.0 | 100.0 |

Clear Conditioner Shampoo

Examples

| | 1 | 2 | 3 |
|---|---|---|---|
| Polyquaternium-10 | 0.5 | 0.5 | 0.5 |
| Sodium laureth sulfate | 9.0 | 9.0 | 9.0 |
| Benzophenone 3 | 1.00 | 1.50 | 0.50 |
| Cocoamidopropylbetaine | 2.5 | 2.5 | 2.5 |
| Dioic acid | 0.05 | 0.15 | 0.1 |
| Iminodisuccinic acid, Na salt | 0.2 | 0.3 | 0.8 |
| Preservative, perfume, thickener, | q.s. | q.s. | q.s. |
| pH adjustment | | | |
| and solubility promoter | | | |
| Water (demineralized) | ad | ad | ad |
| | 100.0 | 100.0 | 100.0 |

The pH is adjusted to 6.

Clear Light Shampoo with Volume Effect

Examples

| | 1 | 2 | 3 |
|---|---|---|---|
| Sodium laureth sulfate | 10.0 | 10.0 | 10.0 |
| Ethylhexyl methoxycinnamate | 2 | 2 | 2 |
| Cocoamidopropylbetaine | 2.5 | 2.5 | 2.5 |
| Dioic acid | 0.05 | 0.1 | 0.01 |
| Disodium EDTA | 0.2 | 0.15 | 0.7 |
| Preservative, perfume, thickener, | q.s. | q.s. | q.s. |
| pH adjustment | | | |
| and solubility promoter | | | |
| Water (demineralized) | ad | ad | ad |
| | 100.0 | 100.0 | 100.0 |

The pH is adjusted to 5.5.

PIT Emulsions

| | Examples | | | | |
|---|---|---|---|---|---|
| | 1 | 2 | 3 | 4 | 5 |
| Glycerol monostearate self- | 0.50 | | 3.00 | 2.00 | 4.00 |
| emulsifying | | | | | |
| Polyoxyethylene(12) cetylstearyl | | 5.00 | | 1.00 | 1.50 |
| ether | | | | | |
| Polyoxyethylene(20) cetylstearyl | | | | 2.00 | |
| ether | | | | | |
| Polyoxyethylene(30) cetylstearyl | 5.00 | | 1.00 | | |
| ether | | | | | |
| Stearyl alcohol | | | 3.00 | | 0.50 |
| Cetyl alcohol | 2.50 | 1.00 | | 1.50 | |
| 2-Ethylhexyl methoxycinnamate | | | | 5.00 | 8.00 |
| 2,4-Bis(4-(2-ethylhexyloxy)-2- | | 1.50 | | 2.00 | 2.50 |
| hydroxy)phenyl)-6-(4- | | | | | |
| methoxyphenyl)(1,3,5)-triazine | | | | | |
| Butyldimethoxydibenzoylmethane | | | 2.00 | | |
| Dimethicodiethylbenzalmalonate | | 6.50 | | | |
| Diethylhexylbutamidotriazone | 1.00 | 2.00 | | 2.00 | |
| Ethylhexyltriazone | 4.00 | | 3.00 | 4.00 | |
| Aminobenzophenone | 1.50 | 4.00 | 3.50 | 5.00 | 2.00 |
| Octocrylene | | 4.00 | | | 2.50 |
| Phenylene-1,4-bis(monosodium) | | | 0.50 | | 1.50 |
| 2-benzimdazyl-5,7-disulfonic acid) | | | | | |
| Phenylbenzimidazolesulfonic acid | 0.50 | | | 3.00 | |
| C₁₂₋₁₅ alkyl benzoate | | 2.50 | | | 5.00 |
| Dicaprylyl ether | | | 3.50 | | |
| Butylene glycol dicaprylate/dicaprate | 5.00 | | | 6.00 | |
| Dicaprylyl carbonate | | | 6.00 | | 2.00 |
| Dimethicone polydimethylsiloxane | | 0.50 | 1.00 | | |
| Phenylmethylpolysiloxane | 2.00 | | | 0.50 | 0.50 |
| Shea butter | | 2.00 | | | 0.50 |
| PVP hexadecene copolymer | 0.50 | | | 0.50 | 1.00 |
| Glycerol | 3.00 | 7.50 | 5.00 | 7.50 | 2.50 |
| Tocopherol acetate | 0.50 | | 0.25 | | 1.00 |
| Dioic acid | 0.10 | 1.00 | 0.20 | 0.50 | 1.50 |
| Diethylhexyl 2,6-naphthalate | | | 2.00 | | |
| Alpha-glucosylrutin | 0.10 | | 0.20 | | |
| DMDM hydantoin | | 0.25 | | 0.60 | 0.45 |
| Parabene | 0.15 | | 0.50 | 0.30 | |
| Konkaben LMB ® | 0.20 | | 0.40 | | |
| Trisodium EDTA | | 0.80 | | | 1.00 |
| Phenoxyethanol | 0.30 | | | 0.20 | 0.50 |
| Ethanol | 3.00 | 2.00 | 1.50 | | 1.00 |
| Perfume | q.s. | qs. | qs. | qs. | q.s. |
| Water | ad | ad | ad | ad | ad |
| | 100 | 100 | 100 | 100 | 100 |

O/W Emulsions

| | Examples | | | | |
|---|---|---|---|---|---|
| | 1 | 2 | 3 | 4 | 5 |
| Glyceryl stearate citrate | | | 2.00 | | 2.00 |
| Glyceryl stearate self-emulsifying | 4.00 | 3.00 | | | |
| PEG-40 stearate | 1.00 | | | | |
| Polyglyceryl-3 methylglucose distearate | | | | 3.00 | |
| Sodium cetearyl sulfate | 0.50 | | | | 1.00 |
| Sorbitan stearate | | | | | 2.00 |
| Stearic acid | | 1.00 | | | |
| Stearyl alcohol | | | 5.00 | | |
| Cetyl alcohol | 3.00 | 2.00 | | 3.00 | |
| Cetyl stearyl alcohol | | | | | 2.00 |
| Caprylic/capric triglyceride | 5.00 | 3.00 | 4.00 | 3.00 | 3.00 |
| Octyldodecanol | | | 2.00 | | 2.00 |
| Dicaprylyl ether | | 4.00 | | 2.00 | 1.00 |
| Paraffinum liquidum | 5.00 | 2.00 | | 3.00 | |
| Zinc oxide | 1.00 | | | | 2.00 |
| Titanium dioxide | | | 1.00 | | |
| Aminobenzophenone | 0.50 | 2.50 | 3.00 | 1.50 | 5.50 |
| 2-Ethylhexyl methoxycinnamate | | 5.50 | | | |
| 2,4-Bis(4-(2-ethylhexyloxy)-2- | 3.00 | | 1.50 | 0.80 | |
| hydroxy)phenyl)-6-(4- | | | | | |
| methoxyphenyl)(1,3,5)-triazine | | | | | |
| Diethylhexylbutamidotriazone | | 2.00 | | | 1.00 |
| Ethylhexyltriazone | 1.80 | | | | |
| Phenylene-1,4-bis(monosodium, 2- | 0.50 | | | | 0.50 |
| benzimidazyl-5,7-disulfonic acid) | | | | | |
| Phenylbenzimidazole sulfonic acid | | 0.50 | | 2.00 | |
| Octocrylene | | | 3.50 | | |
| Butyldimethoxydibenzoylmethane | | | 0.50 | | |
| Dioic Acid | 0.10 | 0.20 | 0.70 | 0.15 | 1.00 |
| Tocopherol | 0.10 | | | | 0.20 |
| Diethylhexyl-2,6-naphthalate | | | 3.50 | | |
| Biotin | | | 0.05 | | |
| Ethylenediaminetetraacetic acid | 0.1 | | 0.10 | 0.1 | |
| trisodium | | | | | |
| Preservative | q.s. | q.s. | q.s. | q.s. | q.s. |
| Polyacrylic acid | 3.00 | 0.1 | | 0.1 | 0.1 |
| Sodium hydroxide solution 45% | q.s. | q.s. | q.s. | q.s. | q.s. |
| Glycerol | 5.00 | 3.00 | 4.00 | 3.00 | 3.00 |
| Butylene glycol | | 3.00 | | | |
| Perfume | q.s. | qs. | qs. | qs. | q.s. |
| Water | ad 100 | ad 100 | ad 100 | ad 100 | ad 100 |
| | Examples | | | | |
| | 6 | 7 | 8 | 9 | 10 |
| Glyceryl stearate citrate | | 2.00 | 2.00 | | |
| Glyceryl stearate self-emulsifying | 5.00 | | | | |
| Stearic acid | | | | 2.50 | 3.50 |
| Stearyl alcohol | 2.00 | | | | |
| Cetyl alcohol | | | | 3.00 | 4.50 |
| Cetyl stearyl alcohol | | 3.00 | 1.00 | | 0.50 |
| C₁₂₋₁₅ alkyl benzoate | | 2.00 | 3.00 | | |
| Caprylic/capric triglyceride | 2.00 | | | | |
| Octyldodecanol | 2.00 | 2.00 | | 4.00 | 6.00 |
| Dicaprylyl ether | | | | | |
| Paraffinum liquidum | | 4.00 | 2.00 | | |
| Cyclic dimethylpolysiloxane | | | | 0.50 | 2.00 |
| Dimethicone polydimethylsiloxane | 2.00 | | | | |
| Titanium dioxide | 2.00 | | | | |
| 4-Methylbenzylidenecamphor | 1.00 | | | | |
| Ethylhexyltriazone | | 3.00 | | | 2.00 |
| Butyldimethoxydibenzoylmethane | 0.50 | | | | 0.50 |
| Aminobenzophenone | 0.50 | 1.50 | 5.00 | 3.30 | 4.00 |
| Dioic acid | 0.30 | 0.10 | 1.00 | 0.50 | 0.10 |
| 2-Ethylhexyl methoxycinnamate | 1.50 | 4.00 | | 2.50 | |
| 2,4-Bis(4-(2-ethylhexyloxy)-2- | 0.80 | | 1.50 | 2.50 | |
| hydroxy)phenyl)-6-(4- | | | | | |
| methoxyphenyl)(1,3,5)-triazine | | | | | |
| Dimethicodiethylbenzalmalonate | | | 6.00 | | |
| Diethylhexylbutamidotriazone | 1.00 | 3.00 | | 2.00 | |
| Octocrylene | 4.00 | | 5.00 | | 3.50 |
| Phenylene-1,4-bis(monosodium, 2- | 0.50 | | 1.00 | | |
| benzimidazyl-5,7-disulfonic acid) | | | | | |
| Phenylbenzimidazole sulfonic acid | | 2.00 | | 1.50 | 0.50 |
| Tocopherol | | | | | 0.10 |
| Ethylenediaminetetraacetic acid | | | 0.20 | | 0.20 |
| trisodium | | | | | |
| Preservative | q.s. | q.s. | q.s. | q.s. | q.s. |
| Xanthan gum | | | 0.20 | | |
| Polyacrylic acid | 0.15 | 0.1 | | 0.05 | 0.05 |
| Sodium hydroxide solution 45% | q.s. | q.s. | q.s. | q.s. | q.s. |
| Glycerol | 3.00 | | 3.00 | 5.00 | 3.00 |
| Butylene glycol | | 3.00 | | | |
| Ethanol | | 3.00 | | 3.00 | |
| Perfume | q.s. | qs. | qs. | qs. | q.s. |
| Water | ad 100 | ad 100 | ad 100 | ad 100 | ad 100 |

W/O Emulsions

| | Examples | | | | |
|---|---|---|---|---|---|
| | 1 | 2 | 3 | 4 | 5 |
| Cetyldimethicone copolyol | | 2.50 | | 4.00 | |
| Polyglyceryl-2 dipolyhydroxystearate | 5.00 | | | | 4.50 |
| PEG-30 dipolyhydroxystearate | | | 5.00 | | |
| Ethylhexyl methoxycinnamate | | 8.00 | | 5.00 | 4.00 |
| 2,4-Bis(4-(2-ethylhexyloxy)-2- | 2.00 | 2.50 | | 2.00 | 2.50 |
| hydroxy)phenyl)-6-(4- | | | | | |
| methoxyphenyl)(1,3,5)-triazine | | | | | |
| Butyldimethoxydibenzoylmethane | | | 2.00 | 1.00 | |
| Diethylhexylbutamidotriazone | 3.00 | 1.00 | | | 3.00 |
| Ethylhexyltriazone | | | 3.00 | 4.00 | |
| 4-Methylbenzylidenecamphor | | 2.00 | | | |
| Aminobenzophenone | 0.50 | 2.50 | 4.50 | 3.00 | 1.80 |
| Octocrylene | 7.00 | 2.50 | 4.00 | | 2.50 |
| Phenylene-1,4-bis(monosodium, 2- | 1.00 | 2.00 | 0.50 | | |
| benzimidazyl-5,7-disulfonic acid) | | | | | |
| Phenylbenzimidazolesulfonic acid | 0.50 | | | 3.00 | 2.00 |
| Dimethicodiethylbenzalmalonate | | | 5.50 | | |
| Titanium dioxide | | 2.00 | 1.50 | | 3.00 |
| Zinc oxide | 3.00 | 1.00 | 2.00 | 0.50 | |
| Paraffinum liquidum | | | 10.0 | | 8.00 |
| C₁₂₋₁₅ alkyl benzoate | | | | 9.00 | |
| Dicaprylyl ether | 10.00 | | | | 7.00 |
| Butylene glycol dicaprylate/dicaprate | | | 2.00 | 8.00 | 4.00 |
| Dicaprylyl carbonate | 5.00 | | 6.00 | | |
| Dimethicone polydimethylsiloxane | | 4.00 | 1.00 | 5.00 | |
| Phenylmethylpolysiloxane | 2.00 | 25.00 | | | 2.00 |
| Shea butter | | | 3.00 | | |
| Diethylhexyl-2,6-naphthalate | | | 6.50 | | |
| PVP hexadecene copolymer | 0.50 | | | 0.50 | 1.00 |
| Octoxyglycerol | | 0.30 | 1.00 | | 0.50 |
| Glycerol | 3.00 | 7.50 | | 7.50 | 2.50 |
| Glycine soya | | 1.00 | 1.50 | | |
| Magnesium sulfate | 1.00 | 0.50 | | 0.50 | |
| Magnesium chloride | | | 1.00 | | 0.70 |
| Tocopherol acetate | 0.50 | | 0.25 | | 1.00 |
| Dioic acid | 0.10 | 0.20 | 0.50 | 0.90 | 1.00 |
| DMDM hydantoin | | 0.25 | | 0.60 | 0.45 |
| Paraben | 0.15 | | 0.50 | 0.30 | |
| Konkaben LMB ® | 0.20 | | 0.40 | | |
| Trisodium EDTA | | 0.80 | | | 1.00 |
| Phenoxyethanol | 0.30 | | | 0.20 | 0.50 |
| Ethanol | 3.00 | 2.00 | 1.50 | | 1.00 |
| Perfume | q.s. | qs. | qs. | qs. | q.s. |
| Water | ad 100 | ad 100 | ad 100 | ad 100 | ad 100 |

Examples

| | 6 | 7 |
|---|---|---|
| Polyglyceryl-2 dipolyhydroxystearate | 4.00 | 5.00 |
| Lanolin alcohol | 0.50 | 1.50 |
| Isohexadecane | 1.00 | 2.00 |
| Myristyl-myristate | 0.50 | 1.50 |
| Petroleum jelly | 1.00 | 2.00 |
| Butyldimethoxydibenzoylmethane | 0.50 | 1.50 |
| Diethylhexylbutamidotriazone | 1.50 | 0.50 |
| Butylene glycol dicaprylate/dicaprate | 4.00 | 5.00 |
| Aminobenzophenone | 2.50 | 4.50 |
| Shea butter | | 0.50 |
| Butylene glycol | | 6.00 |
| Octoxyglycerol | | 3.00 |
| Glycerol | 5.00 | |
| Tocopherol acetate | 0.50 | 1.00 |
| Dioic acid | 0.10 | 0.70 |
| Trisodium EDTA | 0.20 | 0.20 |
| Preservative | q.s. | q.s. |
| Ethanol | | 3.00 |
| Perfume | q.s. | q.s. |
| Water | ad 100 | ad 100 |

Hydrodispersions

| | Examples | | | | |
|---|---|---|---|---|---|
| | 1 | 2 | 3 | 4 | 5 |
| Polyoxyethylene(20) cetylstearyl | 1.00 | | | 0.5 | |
| ether | | | | | |
| Cetyl alcohol | | | 1.00 | | |
| Sodium polyacrylate | | 0.20 | | 0.30 | |
| Acrylate/C10-30-alkyl acrylate | 0.50 | | 0.40 | 0.10 | 0.10 |
| crosspolymer | | | | | |
| Xanthan gum | | 0.30 | 0.15 | | 0.50 |
| 2-Ethylhexyl methoxycinnamate | | | | 5.00 | 8.00 |
| 2,4-Bis(4-(2-ethylhexyloxy)-2- | | 1.50 | 2.00 | | 2.50 |
| hydroxy)phenyl)-6-(4- | | | | | |
| methoxyphenyl)(1,3,5)-triazine | | | | | |
| Dimethicodiethylbenzalmalonate | | 3.50 | | | |
| Butyldimethoxydibenzoylmethane | 1.00 | | 2.00 | | |
| Diethylhexylbutamidotriazone | | 2.00 | | 2.00 | 1.00 |
| Ethylhexyltriazone | 4.00 | | 3.00 | 4.00 | |
| 4-Methylbenzylidenecamphor | | | | | 2.00 |
| Aminobenzophenone | 2.00 | 1.40 | 0.50 | 4.60 | 5.20 |
| Octocrylene | | 4.00 | 4.00 | | 2.50 |
| Phenylene-1,4-bis(monosodium, 2- | 1.00 | | 0.50 | | 2.00 |
| benzimidazyl-5,7-disulfonic acid) | | | | | |
| Phenylbenzimidazolesulfonic acid | 0.50 | | | 3.00 | |
| Titanium dioxide | 0.50 | | 2.00 | 3.00 | 1.00 |
| Zinc oxide | 0.50 | 1.00 | 3.00 | | 2.00 |
| C₁₂₋₁₅ alkyl benzoate | 2.00 | 2.50 | | | |
| Diethylhexyl-2,5-naphthalate | 4.00 | | | | |
| Dicaprylyl ether | | 4.00 | | | |
| Butylene glycol | 4.00 | | 2.00 | 6.00 | |
| dicaprylate/dicaprate | | | | | |
| Dicaprylyl carbonate | | 2.00 | 6.00 | | |
| Dimethicone polydimethylsiloxane | | 0.50 | 1.00 | | |
| Phenylmethylpolysiloxane | 2.00 | | | 0.50 | 2.00 |
| Shea butter | | 2.00 | | | |
| PVP hexadecene copolymer | 0.50 | | | 0.50 | 1.00 |
| Octoxyglycerol | | | 1.00 | | 0.50 |
| Glycerol | 3.00 | 7.50 | | 7.50 | 2.50 |
| Glycine soya | | | 1.50 | | |
| Tocopherol acetate | 0.50 | | 0.25 | | 1.00 |
| Dioic acid | 0.3 | 0.10 | 0.50 | 1.00 | 0.20 |
| DMDM hydantoin | | 0.25 | | 0.60 | 0.45 |
| Parabene | 0.15 | | 0.50 | 0.30 | |
| Konkaben LMB ® | 0.10 | | 0.30 | | |
| Trisodium EDTA | | | 0.70 | | 1.00 |
| Phenoxyethanol | | 0.40 | | 0.20 | 0.50 |
| Ethanol | 3.00 | 2.00 | 1.50 | | 1.00 |
| Perfume | q.s. | qs. | qs. | qs. | q.s. |
| Water | ad | ad | ad | ad | ad |
| | 100 | 100 | 100 | 100 | 100 |

Gel Cream

| | |
|---|---|
| Acrylate/C10-30 alkyl acrylate crosspolymer | 0.40 |
| Polyacrylic acid | 0.20 |
| Xanthan gum | 0.10 |
| Cetearyl alcohol | 3.00 |
| C₁₂₋₁₅-alkyl benzoate | 4.00 |
| Caprylic/capric triglyceride | 3.00 |
| Cyclic dimethylpolysiloxane | 5.00 |
| Dimethicone polydimethylsiloxane | 1.00 |
| Aminobenzophenone | 1.20 |
| Dioic acid | 1.00 |
| Ethylhexyltriazone | 2.00 |
| 2,4-Bis(4-(2-ethylhexyloxy)-2-hydroxy)phenyl)-6-(4- | 1.50 |
| methoxyphenyl)(1,3,5)-triazine | |
| Glycerol | 3.00 |
| Sodium hyroxide | q.s. |
| Preservative | q.s. |
| Perfume | q.s. |
| Water | ad 100.0 |

pH adjusted to 6.0

W/O/W Emulsion

| | |
|---|---|
| Glyceryl stearate | 3.00 |
| PEG-100 stearate | 0.75 |
| Behenyl alcohol | 2.00 |
| Caprylic/capric triglyceride | 8.0 |
| Octyldodecanol | 5.00 |
| C12-15 alkyl benzoate | 3.00 |
| Aminobenzophenone | 1.30 |
| Dioic acid | 1.00 |
| Ethylhexyl methoxycinnamate | 5.00 |
| 2,4-Bis(4-(2-ethylhexyloxy)-2-hydroxy)phenyl)-6-(4- | 1.80 |
| methoxyphenyl)(1,3,5)-triazine | |
| Ethylhexyltriazone | 1.50 |
| Magnesium sulfate (MgSO₄) | 0.80 |
| Ethylenediaminetetraacetic acid | 0.10 |
| Preservative | q.s. |
| Perfume | q.s. |
| Water | ad 100.0 |

pH adjusted to 6.0

Solids-Stabilized Emulsions

| | Examples | | | | |
|---|---|---|---|---|---|
| | 1 | 2 | 3 | 4 | 5 |
| Mineral oil | | | 16.0 | 16.0 | |
| Octyldodecanol | 9.0 | 9.0 | 5.0 | | |
| Caprylic/capric triglyceride | 9.0 | 9.0 | 6.0 | | |
| C₁₂₋₁₅-alkyl benzoate | | | | 5.0 | 8.0 |
| Butylene glycol dicaprylate/dicaprate | | | | | 8.0 |
| Dicaprylyl ether | 9.0 | | | 4.0 | |
| Dicaprylyl carbonate | | 9.0 | | | |
| Hydroxyoctacosanyl hydroxystearate | 2.0 | 2.0 | 2.0 | 2.0 | 1.5 |
| Disteardimonium hectorite | 1.0 | 0.75 | 0.5 | 0.5 | 0.25 |
| Cera microcrystallina + paraffinum liquidum | | | | | 5.0 |
| Hydroxypropylmethylcellulose | | | | | 0.05 |
| Dimethicone | | | | | 3.0 |
| Ethylhexyl methoxycinnamate | | | | | 3.0 |
| 4-Methylbenzylidenecamphor | | | | | 4.0 |
| Diethylhexylbutamidotriazone | | | | | 4.0 |
| Methylene bis-benzotriazolyl- | | | | | 4.0 |
| tetramethylbutylphenol | | | | | |
| Bis-ethylhexyloxyphenol | | 0.5 | | 2.00 | 1.00 |
| methoxyphenyltriazine | | | | | |
| Drometrizole trisiloxane | | 0.50 | | 1.00 | |
| Terephthalidenedicamphorsulfonic | | 1.00 | 0.50 | | 1.50 |
| acid | | | | | |
| Phenyldibenzimidazoletetrasulfonic | | | | 1.50 | 0.5 |
| acid | | | | | |
| Titanium dioxide + alumina + simethicone + aqua | | 2.0 | 4.0 | 2.0 | 4.0 |
| Titanium dioxide + trimethoxycaprylysilane | | | | | 3.0 |
| Zinc oxide | | | | 6.0 | |
| Silica dimethyl silylate | | | 1.0 | | |
| Boron nitride | 2.0 | | | | |
| Starch/sodium metaphosphate | | 0.5 | | | |
| polymer | | | | | |
| Tapioca starch | | | | 1.0 | |
| Dioic acid | 0.80 | 0.10 | 0.40 | 0.50 | 1.00 |
| Aminobenzophenone | 0.40 | 1.80 | 5.00 | 3.50 | 4.00 |
| Sodium chloride | 1.0 | 1.0 | 1.0 | 1.0 | 1.0 |
| Glycerol | 5.0 | 10.0 | 3.0 | 6.0 | 10.0 |
| Trisodium EDTA | | 1.0 | | 1.0 | |
| Methylparaben | 0.21 | | | | 0.2 |
| Propylparaben | 0.07 | | | | |
| Phenoxyethanol | 0.5 | | 0.4 | 0.4 | 0.5 |
| Hexamidine diisethionate | | | | | 0.08 |
| Diazolidinylurea | | | 0.28 | 0.28 | |
| Alcohol | | | | 2.5 | |
| Perfume | q.s. | qs. | qs. | qs. | q.s. |
| Water | ad | ad | ad | ad | ad |
| | 100 | 100 | 100 | 100 | 100 |
| | Examples | | | | |
| | 6 | 7 | 8 | 9 | 10 |
| Mineral oil | | | | | 16.0 |
| Octyldodecanol | 6.0 | | 7.5 | 7.5 | 5.0 |
| Caprylic/capric triglyceride | | | | | 6.0 |
| C12-15 alkyl benzoate | 7.0 | 8.0 | 7.5 | 7.5 | |
| Butylene glycol dicaprylate/dicaprate | 4.0 | 8.0 | | | |
| Dicaprylyl ether | | 8.0 | 7.5 | 7.5 | |
| Dicaprylyl carbonate | 4.0 | | | | |
| Hydroxyoctacosanyl hydroxystearate | 2.0 | 2.0 | 2.0 | 2.0 | 1.5 |
| PVP/hexadecene copolymer | | | | 1.0 | 0.7 |
| Disteardimonium hectorite | 1.0 | 1.0 | 1.0 | 0.5 | 1.0 |
| Dimethicone | | 2.0 | | | |
| Cyclomethicone | | | | 2.0 | |
| Ethylhexyl methoxycinnamate | 5.0 | | 5.0 | | |
| Butylmethoxydibenzoylmethane | | 2.0 | | | 1.0 |
| 4-Methylbenzylidenecamphor | | 4.0 | | | 2.0 |
| Ethylhexyltriazone | 2.0 | 2.0 | | | 1.0 |
| Methylene bis-benzotriazolyl | | | 1.0 | | 0.50 |
| tetramethylbutylphenol | | | | | |
| Dimethicodiethylbenzalmalonate | | 3.80 | | | |
| Bis-ethylhexyloxyphenol | 2.5 | | 1.5 | 3.00 | |
| methoxyphenyltriazine | | | | | |
| Titanium dioxide + alumina + simethicone + aqua | 1.5 | 2.0 | 4.0 | 0.5 | 1.5 |
| Titanium dioxide + trimethoxycaprylylsilane | | | 2.0 | | |
| Zinc oxide | | | 2.0 | | |
| Phenyldibenzimidazoletetrasulfonic | 1.00 | | | 2.00 | |
| acid | | | | | |
| Phenylbenzimidazolesulfonic acid | 2.0 | | | | |
| Diethylhexyl 2,6-naphthalate | | | | | 3.50 |
| Boron nitride | | | | | 0.5 |
| Starch/sodium metaphosphate | 0.5 | | 1.5 | | |
| polymer | | | | | |
| Corn starch modified | | 1.0 | | | |
| Acrylate copolymer | | | | 0.25 | |
| Talc | | | | 2.0 | |
| Sodium chloride | 1.0 | 1.0 | 1.0 | | |
| Dioic acid | 0.10 | 0.80 | 0.60 | 1.50 | 0.40 |
| Aminobenzophenone | 4.80 | 1.00 | 0.50 | 2.50 | 3.50 |
| Magnesium sulfate | | | | | 0.7 |
| Sodium hydroxide solution 45% | 0.5 | 0.5 | | | |
| Glycerol | 5.0 | 7.5 | 5.0 | 10.0 | 3.0 |
| Trisodium EDTA | | 1.0 | 1.0 | | 1.0 |
| Propylene carbonate | 0.33 | 0.33 | 0.33 | | 0.33 |
| Methylparaben | 0.21 | 0.21 | 0.2 | 0.2 | 0.21 |
| Propylparaben | 0.07 | 0.07 | | | 0.07 |
| Phenoxyethanol | 0.5 | 0.5 | 0.5 | 0.5 | 0.5 |
| Hexamidine diisethionate | | | 0.08 | 0.08 | |
| Alcohol | | 5.0 | | | |
| Perfume | q.s. | qs. | qs. | qs. | q.s. |
| Water | ad | ad | ad | ad | ad |
| | 100 | 100 | 100 | 100 | 100 |

1. A cosmetic or dermatological preparation comprising:
a. at least one UV filtering substance and
b. 8-hexadecene-1,16-dicarboxylic acid.

2. The cosmetic or dermatological preparation as claimed in claim 1, wherein the at least one UV filtering substance is present in a total concentration of 0.1% to 30% by weight, based on the total weight of the preparation.

3. The cosmetic or dermatological preparation as claimed in claim 1, wherein the at least one UV filtering substance is present in a total concentration of 0.5 to 20% by weight, based on the total weight of the preparation.

4. The cosmetic or dermatological preparation as claimed in claim 1, wherein the at least one UV filtering substance is present in a total concentration of 1.0 to 15.0% by weight based on the total weight of the preparation.

5. The cosmetic or dermatological preparation as claimed in claim 1, wherein the at least one UV filtering substance is present in a total concentration of less than 20% by weight based on the total weight of the preparation.

6. The cosmetic or dermatological preparation as claimed in claim 1, wherein the 8-hexadecene-1,16-dicarboxylic acid is present in a total concentration of 0.001 to 10% by weight, based on the total weight of the preparation.

7. The cosmetic or dermatological preparation as claimed in claim 1, wherein the 8-hexadecene-1,16-dicarboxylic acid is present in a total concentration of 0.005 to 8% by weight, based on the total weight of the preparation.

8. The cosmetic or dermatological preparation as claimed in claim 1, wherein the 8-hexadecene-1,16-dicarboxylic acid is present in a total concentration of 0.05 to 5% by weight, based on the total weight of the preparation.

9. The cosmetic or dermatological preparation as claimed in claim 1, wherein the UV filtering substance includes at least one hydroxybenzophenone.

10. The cosmetic or dermatological preparation as claimed in claim 9, wherein the at least one hydroxybenzophenone is the aminobenzophenone characterized by the chemical structural formula

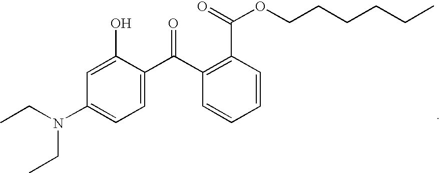

11. The cosmetic or dermatological preparation as claimed in claim 9, wherein the at least one hydroxybenzophenone is present in a total concentration of 0.1 to 20% by weight, based on the total weight of the preparation.

12. The cosmetic or dermatological preparation as claimed in claim 9 wherein the at least one hydroxybenzophenone is present in a total concentration of 0.1 to 15% by weight, based on the total weight of the preparation.

13. The cosmetic or dermatological preparation as claimed in claim 9 wherein the at least one hydroxybenzophenone is present in a total concentration of 0.1 to 10% by weight, based on the total weight of the preparation.

14. The cosmetic or dermatological preparation as claimed in claim 1, wherein the at least one UV filtering substance is selected from the group consisting of triazines, benzotriazoles, UV filters liquid at room temperature, organic pigments and inorganic pigments.

15. The cosmetic or dermatological preparation as claimed in claim 1, wherein the at least one UV filtering substance is selected from the group consisting of dibenzoylmethane derivatives, phenylene-1,4-bis(2-benzimidazyl)-3,3′-5,5′-tetrasulfonic acid bis-sodium salt, 1,4-di(2-oxo-10-sulfo-3-bornylidenemethyl)benzene and salts thereof, and 2,4-bis{[4-(2-ethylhexyloxy)-2-hydroxy]phenyl}-6-(4-methoxyphenyl)-1,3,5-triazine.

16. A cosmetic or dermatological preparation comprising:
a. a hydroxybenzophenone;
b. at least one UV filtering substance selected from the group consisting of triazines, benzotriazoles, UV filters liquid at room temperature, organic pigments and inorganic pigments; and
c. 8-hexadecene-1,16-dicarboxylic acid.

17. A method of treating or preventing undesired aging, pigmentation of the skin, or pigment disorders comprising applying to the skin a cosmetic or dermatological preparation comprising at least one UV filtering substance and 8-hexadecene-1,16-dicarboxylic acid.

18. The method as claimed in claim 17, wherein the at least one UV filtering substance is present in a total concentration of less than 20% by weight based on the total weight of the preparation.

19. The method as claimed in claim 17, wherein the at least one UV filtering substance is present in a total concentration of 0.1 to 15.0% by weight based on the total weight of the preparation.

20. The method as claimed in claim 17, wherein the at least one UV filtering substance includes a hydroxybenzophenone.

21. The method as claimed in claim 20, wherein the hydroxybenzophenone is the aminobenzophenone characterized by the chemical structural formula

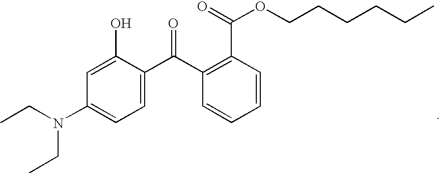

22. The method as claimed in claim 20, wherein the cosmetic or dermatological preparation further comprises at least one UV filtering substance selected from the group consisting of triazines, benzotriazoles, UV filters liquid at room temperature, organic pigments and inorganic pigments.

23. A method of lightening hair or treating or preventing undesired pigmentation of the hair comprising applying a cosmetic or dermatological preparation to the hair comprising at least one UV filtering substance and 8-hexadecene-1,16-dicarboxylic acid.

24. The method as claimed in claim 23, wherein the at least one UV filtering substance is present in a total concentration of less than 20% by weight based on the total weight of the preparation.

25. The method as claimed in claim 23, wherein the at least one UV filtering substance is present in a total concentration of 0.1 to 15.0% by weight based on the total weight of the preparation.

26. The method as claimed in claim 23, wherein the at least one UV filtering substance includes a hydroxybenzophenone.

27. The method as claimed in claim 26, wherein the hydroxybenzophenone is the aminobenzophenone characterized by the chemical structural formula

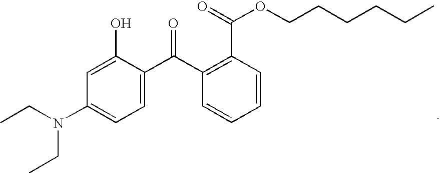

28. The method as claimed in claim 26, wherein the cosmetic or dermatological preparation further comprises at least one UV filtering substance selected from the group consisting of triazines, benzotriazoles, UV filters liquid at room temperature, organic pigments and inorganic pigments.
